(19) United States
(12) Patent Application Publication
(10) Pub. No.: US 2003/0168014 A1
(43) Pub. Date: Sep. 11, 2003

(54) A DEVICE AND AN ASSEMBLY FOR AN ANIMAL RELATED ACTION

(75) Inventors: Adrianus Petrus Hendrikus AARTS, Sprundel (NL); Antoon Peter Andre THEELEN, Maassluis (NL); Karel VAN DEN BERG, Bleskensgraaf (NL); Adriaan Coert VAN LENTEREN, Maasland (NL)

Correspondence Address: HOWREY SIMON ARNOLD & WHITE, CITYPOINT, ONE ROPEMAKER STREET, LONDON, EC2Y 9HS, GB

(73) Assignee: LELY ENTERPRISES AG, Zug (CH)

(21) Appl. No.: 10/249,017

(22) Filed: Mar. 10, 2003

(30) Foreign Application Priority Data
Mar. 11, 2002 (NL) 1020148

(51) Int. Cl. A01J 3/00; A01J 5/00

(52) U.S. Cl. 119/14.02

(57) ABSTRACT

A device provided with measuring means for measuring a characteristic of at least a part of the body of a preferably living animal and for issuing a measurement signal, the device being provided with determining means for determining the fat content of at least a part of the body from the measurement signal.

### CROSS REFERENCE TO RELATED APPLICATIONS

This application claims priority from Dutch Patent No. 1020148, filed 11 Mar. 2002, the contents of which are hereby included by reference in their entirety.

### BACKGROUND OF INVENTION

1. Field of the Invention

The invention relates to devices for automatically measuring a characteristic of the body of a living animal. More particularly it relates to a device for performing an animal related action such as feeding or milking an animal in combination with automatically measuring a characteristic of at least a part of the body of that animal. The invention also relates to a method of performing such measurement in particular, in conjunction with an animal related action.

2. Description of the Related Art

The European patent application EP-A2-1086622 filed 18 May 1993 and hereby incorporated by reference in its entirety, describes a device in the form of a milking box in which a robot arm is disposed that automatically connects teat cups to the teats of the cow present in the milking box. In the floor of the box there are disposed four weighing portions, one for each leg. By means of these weighing portions, the body weight of the cow is determined. There is provided an identification device for recognizing the cow. By means of said identification device, in co-operation with a computer, it is possible to store the weight of a specific cow at several milking runs. Comparison of results of several measurements may provide information for example about the condition of health of an animal. Due to the fact that the weighing device is integrated in a milking box, data about the weight can be obtained in a simple manner several times a day.

A disadvantage of the above-mentioned device is that a sufficiently accurate determination of the condition of health is not possible. In general, the body-score of an animal is considered as a good measure for the condition of health and the production capacity of an animal. Although there does exist a relation between the weight and the body-score, said body-score can not, or very badly, be determined on the basis of the weight of the animal. The fact is that the weight is also influenced by many other factors, such as the degree to which the stomach is filled, the weight of the contents of the bladder and the bowel of the animal, or the weight of a possible fetus. These factors may even vary during the day and disturb a good determination of the body-score.

### SUMMARY OF INVENTION

It would therefore be desirable to provide a device of the above-mentioned type by means of which an accurate determination of the body-score is possible.

To this end, a device for performing a first animal related action, is provided with a measuring device for additionally performing the automatic measurement of a characteristic of at least a part of the body of a living animal and for issuing a measurement signal, the device being further provided with a processing device for determining the fat content of at least a part of the body from the measurement signal. In this way it is possible to determine the fat content of animals that are present in the device for undergoing the animal related action. It is thus possible to combine actions for the measurement and actions for the animal related action, so that the animal will experience less stress. The fat content of an animal, in particular a cow, is an important characteristic for the determination of its condition of health. There is a direct relation with the body-score of the animal.

It is furthermore desirable to provide a device according to the invention with a second action device for automatically performing a second animal related action.

In a further embodiment according to the invention, the device may comprise an animal recognition device for recognizing individual animals. By means of the animal recognition device it is possible to link the measured fat content to a particular individual animal.

In a further advantageous embodiment, the processing device may be provided with a memory, the processing device further comprising a data table for determining the fat content by means of the measurement signal and the processing device being suitable for issuing a fat content signal that is indicative of the determined fat content. For a correct determination of the fat content from the measurement signal obtained, there are required more data, such as breed, age, fat content measured in the past, environmental factors, etc. These factors are stored in a data table. Data from said table can be used in an algorithm that processes the measurement signal for determining the correct fat content. The data table may also comprise data per group of animals. It frequently occurs that a group of animals gives a response that is more or less the same. By comparing the measurement signal with a value of a group of animals from the data table, it is possible to allot to the relevant animal the fat content signal of the corresponding group of animals. The data table may contain data per individual animal. Each animal is different and, therefore, individual data of an animal will be required for an accurate determination of the fat content.

In a further embodiment, the processing device may comprise a comparing device for comparing a momentary fat content determined from a momentary measurement signal with a historical fat content. This makes it possible to indicate whether there is a change in the fat content of an animal or a group of animals. This information is important for a good management of the animal or a group of animals. In some cases said change will be desired; in other cases said change will be undesired because it is an indication of illness or incorrect feeding. In the latter case measures can be taken to adjust the body-score.

In an embodiment according to the invention, the device may comprise an indicating device for indicating the fat content signal. In this way it is possible for an operator to be informed of the fat content of an animal at the location of the device or remotely via a terminal or a mobile telephone.

In another embodiment according to the invention, the device may comprise a warning device for generating a warning, which warning device can be controlled by the fat content signal. In this way it is possible for the operator or a controlling computer to be warned, at the location of the device or remotely, if for example a particular threshold of the fat content signal has been exceeded.

In a further embodiment according to the invention, the device may be controlled by the fat content signal. Depending on the body-score of an animal, the animal has to be treated in a particular manner. In the currently applied management systems, the farmer judges the body-score on the basis of visual observations and palpations carried out on the cow, complemented by external data, if any, such as milk analysis reports. According to the judgement of the body-score, the treatment will be adapted. However, this is very labor-intensive and the actual adaptation often takes place later than desired. By immediately adapting the animal related action in the device to the momentary condition of health or production condition in the form of the body-score, labor is saved and an immediate reaction to a change is possible.

In a further embodiment, the device may comprise a feeding installation for supplying feed. The momentary body-score can be controlled in the direction of the desired body-score by adapting the feed to be supplied. For example, to a cow with a too high body-score, less concentrate or concentrate having a lower energy content should be supplied in the feed trough of the device, in order to reduce her body-score.

In a further advantageous embodiment, the device may comprise a milking machine for automatically performing on the dairy animal a milking related action, such as stimulation, foremilking, pre-cleaning, main milking, post-cleaning and the like. In this way the fat content can be determined automatically at each milking run of the dairy animal. In that case this needs not to be carried out separately by means of a separate installation. If desired, after the momentary production capacity has been determined on the basis of the fat content, the milking machine can be adapted immediately to this new production capacity.

In a specific embodiment, the measuring device measures substances secreted by the animal in relation to the fatty matter metabolism of the animal. If the condition of the animal deteriorates, more fat combustion occurs. This fat combustion can be recognized by specific substances which are then secreted in a higher or lower concentration. Increase or decrease of the physical reserves can be determined by means of detection of the secreted substances, for example by means of breath analysis.

In a particular embodiment according to the invention, the milking machine may be suitable for extracting milk from the dairy animal, and the measuring device measures substances secreted by the animal such substances being milk components, in particular beta-hydroxy butyric acid. An analysis device for analyzing milk is often coupled in a standard way to the milking machine for the purpose of determining the milk quality. For measuring milk components in relation to the fatty matter metabolism, few if any additional provisions have to be provided. Beta-hydroxy butyric acid is a product of the fatty matter metabolism. A high content thereof in the milk will be an indication of fat combustion and of a decrease of the fat content of the animal.

In an advantageous embodiment, an electromagnetic parameter of the animal may be measured by the measuring device. By measuring said parameter, the use of measuring devices comprising few if any moving parts will suffice. This results in a robust embodiment. In particular in an aggressive environment, such as an animal-environment, this is important.

It is advantageous if the measuring device comprises a TOBEC (total body electrical conductivity) meter. This type of meter measures the fat contact in a contactless way, the animal not being hindered by the measurement. These are important conditions for animal-friendly cattle breeding. An example of such a meter is disclosed in U.S. Pat. No. 3,735,247, the contents of which are hereby incorporated by reference in their entirety.

In another embodiment, the measuring device may comprise an electrical impedance meter. Measurement of the electrical impedance can be carried out by means of relatively simple and cheap equipment. Because the measurement is carried out whilst using small currents and low voltages, this measurement does not lead to malfunction of electronically sensitive equipment that may be present around the animal.

In a particular embodiment, the impedance meter may comprise an udder electrode for contact with the udder of the animal. In another particular embodiment, the impedance meter comprises a muzzle electrode for contact with the muzzle of the animal. Both the muzzle and the udder are hardly or not covered with hair and can ensure a good electrical conductivity contact with an electrode without further measures being taken.

It is advantageous to dispose the udder electrode in or on a teat cup. Because, for the purpose of milking the animal, a teat cup is connected to the teat of the animal, no extra mechanical provisions, especially for disposing an electrode on the udder, need to be made.

In a special embodiment, the muzzle electrode may be disposed in or on the feed trough of the feeding installation. During the consumption of feed from the feed trough, the muzzle of the animal will nearly always make contact with the feed trough. It is extra advantageous that the contact point is situated rather constantly at the same place.

In an embodiment according to the invention, the measuring device may comprise a profile meter for measuring at least a part of the profile of the animal. By measuring the profile of the animal and comparing it with standard profiles, the body-score can be determined.

The profile preferably comprises the profile of the upper side of the animal. The condition of the animal is most strongly expressed in the profile of the back of the animal. Apart from this, the upper side of the animal can easily be measured, because it is usually freely accessible from above.

In a preferred embodiment according to the invention, the profile may comprise the profile of the tail portion. Reserve fat is especially stored in the tail portion, so that the profile of the tail portion is a good indicator of the body-score. The tail portion comprises roughly the area around the lumbar vertebras, the ischia and the beginning of the caudal vertebras.

In a further preferred embodiment, the profile may comprise the profile of the shoulder. The shoulder is a place where much reserve fat is stored. An advantage of the shoulder is that it has a round, rather regular shape and that it can easily be measured.

In another embodiment, the profile meter may comprise a camera that is connected to a picture-analyzing device. This has the advantage that the camera, apart from measuring the profile, can also serve as security camera.

In a specific embodiment, the profile meter may comprise a contactless telemeter for measuring the distance from the meter to the surface of the animal. In this way small differences in height in the profile can be detected accurately. This is in particular important for determining the precise shape of the cavities in the area in the vicinity of the tail-head, pin bone, thurl and hip bone. The shape of said cavities is strongly determinative for the body-score. The contactless telemeter can be designed as a laser-Doppler meter or an ultrasonic meter.

In an embodiment according to the invention, the profile meter may comprise one or more feeler devices. A measurement by means of feeler devices is not disturbed by the effect of shadow or the reflection condition of the skin. Due to this, the measurement is less sensitive to environmental factors.

A particular embodiment of a device according to the present invention is characterized in that the profile meter comprises a bag to be filled with a fluid, which bag is provided with a fluid measuring device for measuring the surface related contents of the bag. If this bag is disposed on the back of the animal to be measured, the bag will assume the shape of the profile of the back. The total outer surface of the bag remains equal to the outer surface of the bag in its condition of rest. However, its volume will change. An advantage of this profile measurement is that it can be carried out in a cheap and robust manner.

In a special embodiment, the profile meter may comprise a stop for keeping the profile meter in a fixed position relative to the body of the animal. In this way the surface related contents of the bag, which is a measure for the profile, is always determined relative to the same position of an animal. Consequently, different profile measurements of an animal can be compared with each other in a reliable manner.

It is advantageous if the fluid measuring device comprises a volume meter. Volume changes can be measured in a relatively simple and cheap manner.

In another advantageous embodiment, the fluid measuring device may comprise a flow meter. By means of said flow meter it is possible accurately to indicate changes in the profile when the bag is moved over a profile.

In a particular embodiment according to the invention, there is provided a heating element in the fluid for keeping the fluid at a constant temperature. A constant temperature in the region of the body temperature of the animal is necessary for making the contact with the animal as pleasant as possible. Moreover, by means of this measure it is possible to prevent the fluid from freezing or coagulating.

The fluid is in particular a liquid because of the fact that liquids can be measured more easily than gases.

In an embodiment according to the invention, the profile meter may be connected to a following device for following movements of the animal. For this purpose, the profile meter follows for example the device for following the rear of the animal or a robot arm of the milking machine. Thus the measurement is not disturbed by movements of the animal.

It is advantageous if the profile meter is movable relative to the animal. This makes it possible to determine the profile of a large surface in an accurate and controlled manner.

According to a further advantageous embodiment of the invention, the device may further be provided with a weighing device for determining the weight of the living animal. By combining the values of weight and fat content of the animal, a more complete picture of the condition of the animal may be determined. Such a weighing device may be for instance of the type disclosed in EPA second action device for performing a second animal related action may be controllable by the fat content signal. It frequently occurs that a certain amount of feed is supplied in the device for the first animal related action. The latter device is optimally utilized if it is kept occupied for the first animal related action. Then, the planned amount of feed to be supplied may not always be consumed. In that case it is advantageous if the remaining amount can be supplied in a second feeding installation.

According to a further aspect of the present invention, there is disclosed a method of feeding a dairy animal comprising the steps of: performing an automatic milking related action on the dairy animal; determining the fat content of at least a part of the body of the dairy animal while performing the automatic milking related action; and providing feed to the dairy animal on the basis of the determined fat content.

According to a still further aspect of the present invention, there is disclosed a method of milking a dairy animal comprising the steps of: providing an automatic milking machine for automatically performing a milking related action on the dairy animal; determining the fat content of at least a part of the body of the dairy animal; and milking the dairy animal on the basis of the determined fat content. Such methods may be performed using the device disclosed above.

According to the present invention there is also disclosed a profile meter for the measurement of a characteristic of at least a part of the body of a living animal, the meter comprising a bag to be filled with a fluid, the bag being provided with a fluid measuring device in communication with the interior of the bag for measuring an amount of fluid displaced from the bag and for issuing a measurement signal.

Various animal related actions may be considered within the context of the present invention as first or second animal related actions. Such actions may include without limitation: milking and other milking related actions such as stimulation, foremilking, pre-cleaning and post-cleaning; feeding and feeding related actions; identifying; weighing; grooming; treating; and other measurement and diagnostic practices that may be required as part of the animal husbandry.

### BRIEF DESCRIPTION OF DRAWINGS

Embodiments of a device according to the invention will be described hereinafter by way of example only with reference to the drawings, in which:

FIG. 1. is a plan view of a device according to one embodiment of the invention with a cow present therein.

FIG. 2 is a detail of the rear side of a device according to the invention provided with a profile meter in the form of feeler devices.

FIG. 3 is a detail of the rear side of a device according to the invention provided with a profile meter in the form of a bag.

### DETAILED DESCRIPTION

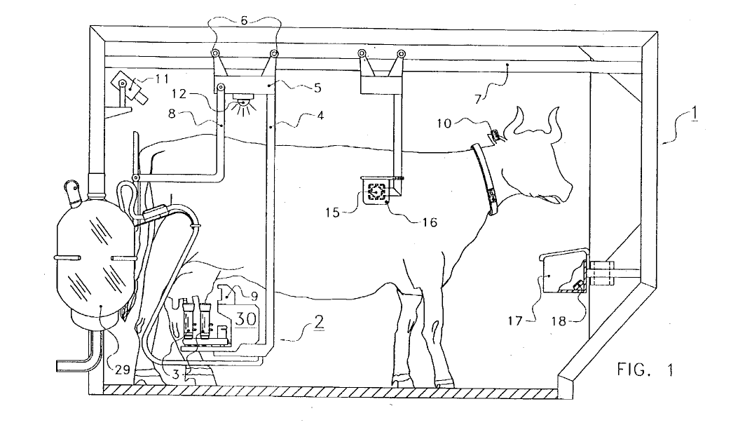

FIG. 1 is an overall picture of one embodiment of a device according to the invention. The device is included in a non-shown cow area, such as a shed, where cows can move about freely. The basis of the device is constituted by a box-shaped frame 1 that serves as fastening place for appendages and for positioning a cow to be milked. A robot arm 2 with milking equipment is provided in the device for automatically connecting teat cups 3 to the udder of the cow. The robot arm 2 is connected, via a vertical connecting element 4, to an arm suspension 5. By means of a drive and wheels 6, said arm suspension 5 is movable over a rail 7 that constitutes part of the frame 1. A device 8 for following the rear of the animal is fitted to the arm suspension 5. Said device 8 for following the rear of the animal is continuously in contact with the rear of the animal, so that movements of the animal in the longitudinal direction of the device are observed by sensors and the drive of the arm suspension 5 can be controlled accordingly. Teat cups 3 are connected to the arm for the purpose of milking the animal. The teat cups 3 can be connected automatically to the teats. There is provided a laser 9 for locating the teats. There is provided a milk glass 29 for collecting the milk from the teat cups before discharging the milk to a storage unit. Each cow is provided with a transponder 10 so as to be able to be identified by the device.

A camera 11 is disposed on the frame 1 for taking a picture of the animal. The camera 11 is focused in particular on the rear part of the body. The camera 11 is disposed movably, so that the position in which the picture is taken can be adapted to the dimensions of the animal. A computer is connected to the camera 11 for analyzing the pictures taken. The picture-analyzing program of the computer recognizes in each digital picture characteristic points of the animal, such as the position of the hip bones and the spine. Said points are used for determining the profile.

In an alternative embodiment, the profile is determined with the aid of the shadow of the animal as described in the international application WO 99/33022 designating the United States, the contents of which are hereby incorporated by reference in their entirety.

An ultrasonic meter 12 is fastened to the arm suspension 5 so as to be positioned above the back of the animal. Because the arm suspension 5 follows the cow, the ultrasonic meter 12 will always be positioned above a specific place on the back. The ultrasonic meter 12 measures the distance from the meter to a point on the back by emitting and receiving ultrasonic sound waves. The profile is determined by measuring the distance to several points.

Instead of an ultrasonic meter, a laser-Doppler meter may be used. The principle of operation remains the same: instead of sound waves, laser beams are emitted in the direction of the animal, reflected and received again. The distance is determined from the comparison of the emitted beams with the reflected beams. In particular the depth of the cavities in the area in the vicinity of the tail-head, pin bone, thurl and hip bone constitutes an important value.

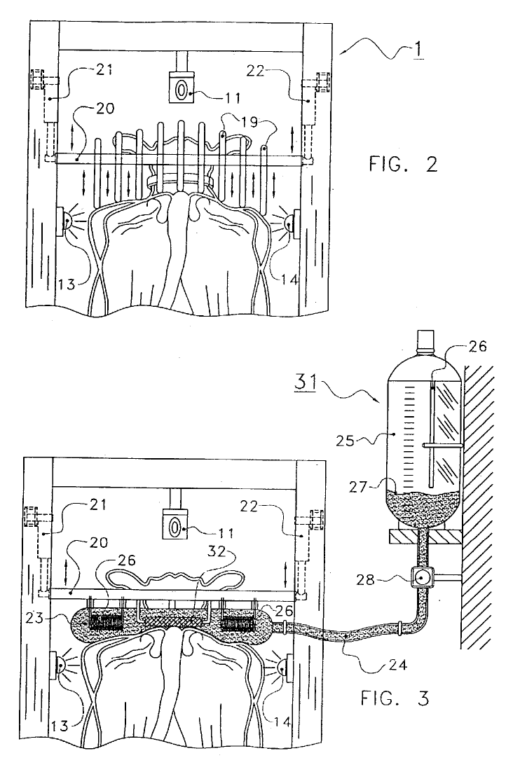

In the embodiment of FIG. 2 a laser-Doppler meter 13 and an ultrasonic meter 14 are fastened to the lateral side of the frame 1 for making it possible also to measure the profile from the lateral side.

A TOBEC-meter 15 is fitted to the rail 7 for the purpose of measuring the fat content directly on the body of the cow. The principle of operation is described in detail in U.S. patent application U.S. Pat. No. 3,735,247. There is provided a protecting element 16 around the TOBEC-meter 15 for protecting other electronic equipment against the electromagnetic radiation emitted by the meter. The TOBEC-meter 15 is suspended from the rail 7 for the purpose of being guided along the animal, independently of the animal or the robot arm 2.

There is provided a feed trough 17 for supplying feed. The controlling computer determines the desired amount and the desired type of feed that is supplied to the animal. A metal muzzle electrode 18 is provided in the feed trough 17. When the animal is consuming feed from the feed trough 17, its muzzle will make contact from time to time with the muzzle electrode 18. In a teat cup 3 there is provided a teat electrode for making contact with the teat. When the teat cup 3 has been connected, an alternating current can be applied over both electrodes. The animal will constitute a conductive connection between the muzzle electrode 18 and the teat electrode. A current meter subsequently measures the current intensity, after which the controlling computer determines the animal impedance (bio-electrical impedance, BIA) of the animal from the current intensity. Said animal impedance can be coupled to the total fat content of the animal. For this purpose, the controlling computer compares the measurement signal with values from a data table. In order to prevent leakage currents, the trough is electrically isolated from the frame 1.

In a non-shown embodiment, there is provided a drinking trough with water. An electrode is also provided in said drinking trough. When the cow puts her muzzle into the water to drink, the water will constitute the electrical connection between the muzzle and the electrode. This embodiment has the advantage that the water always makes a good contact with the muzzle.

The fat content signal that is obtained by means of BIA or TOBEC-measurement relates to the entire body or a major part of the body. For a good evaluation of said signal it is necessary to take data about inter alia the size of the fetus and the stage of pregnancy into account.

FIG. 2 shows an alternative embodiment in which the profile of the rear part of the animal is determined by means of a set of feeler devices 19. These are disposed on a carrier beam so as to be situated side by side in the latitudinal direction. Via two cylinders 21, 22, said carrier beam 20 is movable in height as a whole relative to the frame 1. The feeler devices 19 are individually movable in height, such that they adapt themselves to the profile of the back of the animal. The height of each feeler device 19 is converted into an electrical measurement signal by a potentiometer that is fastened to the feeler device. The controlling computer processes the measurement signals from each feeler device 19 into a profile. The carrier beam 20 is movable as a whole in the longitudinal direction of the device by being guided over a rail 7. This makes it possible always to measure the most characterizing points of an animal for determining the body-score, in particular the cavities in the area in the vicinity of the tail-head, pin bone, thurl and hip bone of a cow, irrespective of the dimensions of the cow. For being guided smoothly over the back of the animal, the contact surface of a feeler device 19 consists of a rotatable element.

In a non-shown embodiment, instead of feeler devices, an ultrasonic meter is fastened to the carrier beam 20. By means of the cylinders 21 and 22, said ultrasonic meter is brought against the body of the cow, preferably in the vicinity of the hip bones. A good measurement requires a good contact with the skin. By means of picture analysis, the fat content is determined from the measured digital ultrasonic picture of the subcutaneous layers. The principle of this measurement is described in detail in U.S. patent publication U.S. Pat. No. 5,960,105, the contents of which are hereby incorporated by reference in their entirety.

FIG. 3 shows another embodiment for measuring the profile of the tail portion of a cow. The carrier beam 20 is provided with a bag 23 filled with a liquid, such as water. Via a flexible tube, said bag 23 is connected to a measuring glass 25 that is located at a higher level than the bag 23 and that is partially filled with the liquid. The carrier beam 20 is connected to cylinders 21, 22, so that the bag 23 can be moved in vertical direction. When an animal is present in the device, the bag 23 is brought from the position of rest above the animal onto the back of the animal. For this purpose, a stop 32 is fastened to the carrier beam, which stop 32 will rest on the spine of the animal. The stop 32 is designed as a roll, so that the animal can move easily in the longitudinal direction of the device relative to the profile meter. The bag 23 will assume a shape that is in accordance with the profile of the animal. According to the profile of the animal, the bag 23 will assume another shape and will have a greater or smaller contents, the so-called surface related contents. A smaller respectively a greater surface related contents will ensure that more respectively less liquid will be present in the measuring glass 25. The amount of liquid in the measuring glass 25 will thus form a measure for the profile and consequently for the fat content of the animal. Electrodes 26 are provided in the measuring glass 25 for determining the height of the liquid level 27. The controlling computer compares said measured height with data from a data table. Said data have been obtained from previous measurements and deviations therefrom give an indication of a change of the fat content. It is desirable that the measurements on the animal are carried out each time at exactly the same place. Due to the fact that the profile meter is coupled to the device 8 for following the rear of the animal, it is possible to measure each time at the same length co-ordinate. The stop 32 ensures that for each cow and for each measurement the same reference height is applied. There is also provided a flow meter 28 in the tube 24. By integration of the measured volume flow over the measurement time it is possible to determine the total change of volume.

There is provided a heating coil 26 in the bag for the purpose of heating the measuring liquid. This makes it possible to heat the liquid until it has reached a temperature that is pleasant to the animal. This also prevents the measuring liquid from freezing. Freezing can also be prevented by adding antifreeze to the liquid.

Many modifications in addition to those described above may be made to the structures and techniques described herein without departing from the spirit and scope of the invention. Accordingly, although specific embodiments have been described, these are examples only and are not limiting upon the scope of the invention.

1. A device for performing a first animal related action, the device being provided with a measuring device for additionally performing the automatic measurement of a characteristic of at least a part of the body of a living animal and for issuing a measurement signal, the device being further provided with a processing device for determining the fat content of at least a part of the body from the measurement signal.

2. A device as claimed in claim 1, characterized in that the device comprises a second action device for automatically performing a second animal related action.

3. A device as claimed in claim 1, characterized in that the device further comprises an animal recognition device for recognizing individual animals.

4. A device as claimed in claim 1, characterized in that the processing device is provided with a memory, the processing device further comprising a data table for determining the fat content by means of the measurement signal and the processing device issuing a fat content signal that is indicative of the determined fat content.

5. A device as claimed in claim 4, characterized in that the data table comprises data per group of animals.

6. A device as claimed in claim 4, characterized in that the data table comprises data per animal.

7. A device as claimed in claim 1, characterized in that the processing device comprises a comparing device for comparing a momentary fat content determined from a momentary measurement signal with a historical fat content.

8. A device as claimed in claim 4, characterized in that the device comprises an indicating device for indicating the fat content signal.

9. A device as claimed in claim 4, characterized in that the device comprises a warning device for generating a warning, which warning device can be controlled by the fat content signal.

10. A device as claimed in claim 4, characterized in that the device can be controlled by the fat content signal.

11. A device as claimed in claim 1, characterized in that the device comprises a feeding installation for supplying feed.

12. A device as claimed in claim 1, the animal being a dairy animal, characterized in that the device comprises an automatic milking machine and the animal related action performed is a milking related action, selected from the group consisting of stimulation, foremilking, pre-cleaning, main milking and post-cleaning.

13. A device as claimed in claim 1, characterized in that the measuring device measures substances secreted by the animal as a result of the fatty matter metabolism of the animal.

14. A device as claimed in claim 12, characterized in that the milking machine extracts milk from the dairy animal, and in that the measuring device measures substances secreted by the animal, such substances being milk components, in particular beta-hydroxy butyric acid.

15. A device as claimed in claim 14 characterized in that the measuring device measures beta-hydroxy butyric acid.

16. A device as claimed in claim 1, characterized in that the measuring device measures an electromagnetic parameter of the animal.

17. A device as claimed in claim 15, characterized in that the measuring device comprises a TOBEC-meter.

18. A device as claimed in claim 15, characterized in that the measuring device comprises an electrical impedance meter.

19. A device as claimed in claim 12, characterized in that the measuring device comprises an impedance meter comprising an udder electrode for contact with the udder of the animal.

20. A device as claimed in claim 19, characterized in that the automatic milking machine is provided with a teat cup and the udder electrode is disposed in or on the teat cup.

21. A device as claimed in claim 18, characterized in that the impedance meter comprises a muzzle electrode for contact with the animal.

22. A device as claimed in claim 21, characterized in that the device comprises a feed trough and the electrode is disposed in or on the feed trough.

23. A device as claimed in claim 1, characterized in that the measuring device comprises a profile meter for measuring at least a part of the profile of the animal.

24. A device as claimed in claim 23, characterized in that the profile comprises the profile of the upper side of the animal.

25. A device as claimed in claim 23, characterized in that the profile comprises the profile of the tail portion.

26. A device as claimed in claim 23, characterized in that the profile comprises the profile of the shoulder.

27. A device as claimed in claim 23, characterized in that the profile meter comprises a camera that is connected to a picture-analyzing device.

28. A device as claimed in claim 23, characterized in that the profile meter comprises a contactless telemeter for measuring the distance from the meter to the surface of the animal.

29. A device as claimed in claim 28, characterized in that the telemeter comprises a laser meter.

30. A device as claimed in claim 23, characterized in that the profile meter comprises one or more feeler devices.

31. A device as claimed in claim 23, characterized in that the profile meter comprises a bag to be filled with a fluid, which bag is provided with a fluid measuring device for measuring the surface related contents of the bag.

32. A device as claimed in claim 31, characterized in that the profile meter comprises a stop for keeping the profile meter in a fixed position relative to the body of the animal.

33. A device as claimed in claim 31, characterized in that the fluid measuring device comprises a volume meter.

34. A device as claimed in claim 31, characterized in that the fluid measuring device comprises a flow meter.

35. A device as claimed in claim 31, characterized in that there is provided a heating element in the fluid for keeping the fluid at a constant temperature.

36. A device as claimed in claim 31, characterized in that the fluid is a liquid.

37. A device as claimed in claim 23, characterized in that the profile meter is connected to a following device for following movements of the animal.

38. A device as claimed in claim 23, characterized in that the profile meter is movable relative to the animal.

39. A device as claimed in claim 4 wherein the device is in communication with a second device for performing a second animal related action, characterized in that the second device can be controlled by the fat content signal.

40. A method of feeding a dairy animal comprising the steps of:
performing an automatic milking related action on the dairy animal;
determining the fat content of at least a part of the body of the dairy animal while performing the automatic milking related action; and
providing feed to the dairy animal on the basis of the determined fat content.

41. The method according to claim 40, wherein the step of providing feed to the dairy animal takes place during milking of the dairy animal.

42. The method according to claim 40 further comprising the step of providing a feed installation and the step of providing feed to the dairy animal takes place at least partially at the feeding installation.

43. A method of milking a dairy animal comprising the steps of:
providing an automatic milking machine for automatically performing a milking related action on the dairy animal;
determining the fat content of at least a part of the body of the dairy animal; and
milking the dairy animal on the basis of the determined fat content.

44. A profile meter for the measurement of a characteristic of at least a part of the body of a living animal, the meter comprising a bag to be filled with a fluid, the bag being provided with a fluid measuring device in communication with the interior of the bag for measuring an amount of fluid displaced from the bag and for issuing a measurement signal.

45. A meter as claimed in claim 44, characterized in that the profile meter comprises a stop for keeping the profile meter in a fixed position relative to the body of the animal.

46. A meter as claimed in claim 44, characterized in that the fluid measuring device comprises a volume meter.

47. A meter as claimed in claim 44, characterized in that the fluid measuring device comprises a flow meter.

48. A meter as claimed in claim 44, characterized in that there is provided a heating element in the fluid for keeping the fluid at a desired temperature.

49. A meter as claimed in claim 44, characterized in that the fluid is a liquid.

50. A meter as claimed in claim 44, characterized in that the profile meter is connected to a following device for following movements of the animal.

51. A meter as claimed in claim 44, characterized in that the profile meter is movable relative to the animal.

52. A meter as claimed in claim 44, the device being further provided with a processing device for determining the fat content of at least a part of the body from the measurement signal.
